(19) United States
(12) Patent Application Publication
(10) Pub. No.: US 2022/0152253 A1
(43) Pub. Date: May 19, 2022

(54) Self-Adjustable Germicidal Irradiation Apparatus And Method

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/137,763

(22) Filed: Dec. 30, 2020

Related U.S. Application Data
(63) Continuation-in-part of application No. 17/099,271, filed on Nov. 16, 2020.

(51) Int. Cl. A61L 2/24; A61L 2/10

(52) U.S. Cl. CPC A61L 2/24; A61L 2202/14; A61L 2/10

(57) ABSTRACT

A self-adjustable germicidal irradiation apparatus includes a radiant source, a driver, and a controller. The driver converts an external power to an internal power to activate the radiant source. The connects to the driver and is configured to adjust a radiant power emitted by the radiant source. The radiation source is configured to generate a wavelength in an ultraviolet (UV) wavelength range 190˜400 nm. The controller is configured automatically to limit a UV dosage received by an object exposed to the UV radiation of the radiant source, such that the received UV dosage of the object does not exceed a UV threshold limit value (TLV) dosage defined by American Conference of Governmental Industrial Hygienists (ACGIH). A related self-adjustable germicidal irradiation method is also proposed.

### BACKGROUND

### Technical Field

The present disclosure is part of a Continuation-in-Part (CIP) of US Patent Application No. 17/099,271, filed 16 November 2020, the content of which being incorporated by reference in its entirety.

Description of Related Art

In US Patent Application No. 17/099,271, a germicidal light dosage dispensing system was introduced. The system includes at least one light source, one driver for each at least one light source, and a germicidal light dosage dispensing mechanism. The at least one light source is a germicidal light source emitting a light with a spectral power distribution (SPD) >90% in the wavelength range 190˜420 nm. The driver converts an external power to an internal power for activating the at least one light source and is controllable by the germicidal light dosage dispensing mechanism. The germicidal light dosage dispensing mechanism limits the total dispensed dosage emitted by the at least one light source over an 8-hour period to be less than the ACGIH defined ultraviolet (UV) Threshold Limit Value (TLV) dosage. Additionally, the germicidal light dosage dispensing mechanism is configured to operate the light source intermittently or continuously, and the germicidal light dosage dispensing mechanism is configurable according to a distance between the light source and a surface to be disinfected. More specifically, the germicidal light dosage dispensing mechanism has at least two radiant power settings for the light source, wherein a lower radiant power setting of the at least two radiant power settings is used when the light source is closer to the surface to be disinfected, and wherein a higher radiant power setting of the at least two radiant power settings is used when the light source is farther from the surface to be disinfected.

The examples given in U.S. patent application Ser. No. 17/099,271 illustrate pre-configured settings for different intermittent operation times based on the mounting height of the light source for delivering different UV dosages to a surface (FIG. 4). This may lead to the interpretation that the original application is applicable only to a limited number of pre-configured settings or that the settings must be performed manually by a user, though the inventors do not suggest or imply neither restriction. This CIP application expands the original application by proposing a self-adjustable germicidal irradiation apparatus that may adjust the germicidal irradiation dosage automatically, i.e., without manual intervention (or a pre-configuration) by a user. A related self-adjustable germicidal irradiation method is also proposed.

### SUMMARY

American Conference of Governmental Industrial Hygienists (ACGIH) has published a UV Safety Guidelines as shown in FIG. 1 (ACGIH ISBN: 0-9367-12-99-6). It shows the UV Threshold Limit Values (TLVs), which is the maximum allowable dosage (in mJ/cm²) for each UV wavelength over an 8-hour period. For example, the TLV for 222 nm wavelength is set to 22 mJ/cm². It is noted that each wavelength has a different TLV dosage limit.

The radiant power is the radiant energy emitted by a radiant source and is measured in milli-Watts or mW. The irradiance is defined as the radiant energy per unit area, measured in mW/cm². The germicidal irradiation dosage, or the UV dosage, or simply the Dosage, can be defined as:

Dosage (mJ/cm²)=Irradiance (mW/cm²)×Time (second)

From this definition, the germicidal irradiation dosage depends on two factors: the irradiance and the time (of exposure under a given irradiance). To dispense a certain germicidal irradiation dosage, e.g., 5 mJ/cm², one can use a high radiant power radiant source (resulting in a higher irradiance) with a short exposure time or use a low radiant power radiant source (resulting in a lower irradiance) with a longer exposure time. Therefore, it is reasonable to manipulate appropriately these two factors, the irradiance and the exposure time, without exceeding the ACGHI UV Safety Guidelines.

In one aspect, the self-adjustable germicidal irradiation apparatus comprises a radiant source, a driver, and controller. The driver converts an external power to an internal power to activate the radiant source, and the controller connects to the driver and is configured to adjust a radiant power emitted by the radiant source, e.g., through adjusting the output wattage of the driver. The radiation source is configured to generate a wavelength in a wavelength range 190˜400 nm. Moreover, the controller is configured automatically to limit a UV dosage received by an object exposed to the UV radiation of the radiant source, such that the received UV dosage of the object when extrapolated over an eight-hour period does not exceed a UV threshold limit value (TLV) dosage defined by American Conference of Governmental Industrial Hygienists (ACGIH) for the wavelength.

Consider a radiant source that emits only 222 nm wavelength continuously. If the controller limits the radiant source to emit less than 22 mJ/cm²/(8 hours×60 minutes×60 seconds)×1000=0.7639 μJ/cm² per second at distance zero, then no object exposed to the irradiation of this radiant source would exceed ACGIH TLV limit for the 222 nm wavelength over eight-hour period. Take another example where a 222 nm radiant source emits less than 22 mJ/cm²/8 hours=2.75 mJ/cm² every hour at the top of the hour at distance zero (i.e., emitting 222 nm wavelength intermittently). Then no object exposed to the irradiation of this second radiant source would exceed ACGIH TLV limit for the 222 nm wavelength over eight-hour period.

Assume the proposed apparatus with a 222 nm radiant source is mounted at a 10 ft ceiling in a room, a desk is the room has a height of 2.5 ft, and the height of an occupant in sitting position is 4.5 ft. Then the controller may limit the received 222 nm wavelength dosage dispensed continuously to be less than 0.7639 μJ/cm² per second at a distance 10 ft-4.5 ft=5.5 ft, so the occupant will never receive any 222 nm dosage beyond the ACGIH TLV over an eight-hour period. The desk will receive a 222 nm wavelength dosage much lesser than 0.7639 μJ/cm² for it is 10 ft-2.5 ft=7.5 ft away from the radiant source, thus resulting insufficient surface germicidal irradiation. When there are no occupants in the room, it is preferrable for the controller to increase the radiant power of the radiant source for providing an adequate germicidal irradiation to the desk 7.5 ft away from the radiant source. When an occupant returns to the room, the controller may resume the UV dosage dispensing for a 5.5 ft distance. To toggle between two germicidal irradiation dosage based on occupancy can be achieved by using an occupancy sensor. In some embodiments, the apparatus further comprises an occupancy sensor. The controller is configured to toggle between two UV dosages received by an object exposed to the UV radiation of the radiant source, depending on whether there is a motion detection by the occupancy sensor. It is foreseeable for a controller to automatically toggle the UV dosage emitted by the radiant source according to a schedule, e.g., an off-hour/work-offer schedule, without any manual intervention by a user.

There are different radiant sources that could be used to emit a wavelength in an UV wavelength range 190˜400 nm. A radiant source may have one peak wavelength, or more than one peak wavelengths, or no peak wavelength at all in a wavelength range 190˜400 nm. An excimer lamp with one type of gas may have only one peak wavelength. When mixing with two types of gas, an excimer lamp may have two peak wavelengths. A light emitting diode (LED) radiant source may not have an obvious peak wavelength. When there is only one peak wavelength, the configuration of the controller may be simplified according to the peak wavelength dosage emitted by the radiant source, since the UV dosages from other UV wavelengths are negligible. In some embodiments, the radiation source has one peak wavelength in a wavelength range 190˜400 nm, and the controller is configured to limit a peak-wavelength UV dosage received by an object exposed to the UV radiation of the radiant source not exceeding a UV TLV dosage calculated based on the ACGIH Safety Guidelines at the peak wavelength. The example discussed above uses a radiant source with a peak wavelength at 222 nm.

When a radiant source has more than one peak wavelengths in the 190˜400 nm range, it may not be adequate to consider the ACGIH TLV compliance on each peak wavelength individually. Consider a hypothetical radiant source that has two peak wavelengths, one at 222 nm and another at 254 nm. Assume a controller is configured to have this hypothetical radiant source emitting UV dosage such that an object will receive 90% of the ACGIH TLVs at both 222 nm and 254 nm over an 8-hour period. In this case, even though the 222 nm dosage and the 254 nm dosage does not exceed the ACGIH TLV limit individually, but their combined UV dosage is considered exceeding the ACGIH UV Safety Guidelines. It is proposed to use the sum of the ratio of received UV dosage by an object to the ACGIH TLV dosage for every wavelength in the 190˜400 nm range to determine whether the combined dosage of all UV wavelengths would exceed the ACGIH TLV limit or not. Therefore, in some embodiments, the radiation source has multiple wavelengths in a wavelength range 190˜400 nm, and the controller is configured to limit the sum of the ratio of the received UV dosage extrapolated over 8-hour period by an object to the ACGIH TLV dosage for every wavelength in the 190˜400 nm range to be less than 100%. This condition can be represented mathematically in the following formula:

$${\sum }_{i = 190 nm}^{400 nm} \frac{Received Dosage Extropolated over 8 hours at wavelength i}{ACGIH TLV at wavelength i} < 100 \%$$

It can be seen from this formula that when a UV dosage at any wavelength in 190˜400 nm range exceeds the corresponding ACGIH TLV at that wavelength, then the UV dosage of the whole apparatus is considered exceeding the ACGIH UV Safety Guidelines, which is a reasonable conclusion. Moreover, when the radiant source has only one peak wavelength in a wavelength range 190˜400 nm, the formula above can be simplified by neglecting the contribution of non-peak wavelengths as the following:

$$\frac{Rec eived Dosage Extropolated over 8 hours at the paeakwavelength}{ACGIH TLV at the peak wavelength} < 100 \%$$

Consider another example of the hypothetical radiant source with two peak wavelengths, 222 nm and 254 nm. If the controller is configured to limit the UV emission of the hypothetical radiant source such that an object receives less than 50% of the ACGIH TLV for either 222 nm and 254 nm wavelengths over an 8-hour period, then the combined UV exposure (less than 50%+50%) is regarded as not exceeding the ACGIH UV Safety Guidelines. Similarly, if the controller is configured to limit the UV emission of the hypothetical radiant source such that an object receives UV exposure less than 60% of the ACGIH TLV at 222 nm and less than 40% of the ACGIH TLV at 254 nm wavelengths over an 8-hour period, then the combined UV exposure (less than 60% +40%) is still regarded as not exceeding the ACGIH UV Safety Guidelines. However, if the received UV exposure is 55% at 222 nm and 55% at 254 nm, thus yield a combined total exposure of 55%+55%=110%, then the combined exposure is considered exceeding the ACGIH UV Safety Guidelines.

When a radiant source emits a UV wavelength continuously, the condition states above can be simplified as:

$${\sum }_{i = 190 nm}^{400 nm} \frac{Received Dosage at wavelength i per second}{ACGIH TLV at wavelength i per second} < 100 \%$$

Therefore, in some embodiments, the controller is configured to operate the radiant source continuously at a same radiant power, the controller is configured to limit the sum of the ratio of the per-second UV dosage received by an object to the per-second ACGIH TLV dosage for each wavelength in the 190˜400 nm range to be less than 100%. The per-second ACGIH TLV for 222 nm, again, can be calculated by: 22 mJ/cm²/(8 hours×60 minutes×60 seconds)×1000=0.7639 μJ/cm² per second

For a UV radiant source having one peak wavelength, the formula above can be reduced to the following by neglecting other non-peak wavelengths:

$$\frac{Received Dosage at the paeakwavelength per second}{ACGIH TLV at the peak wavelength per second} < 100 \%$$

Therefore, in some embodiments, where the radiation source has one peak wavelength in a wavelength range 190˜400 nm, the controller is configured to operate the radiant source continuously, and the controller is configured to limit the per-second UV dosage received by an object not to exceed the per-second ACGIH TLV dosage at the peak wavelength. Consider a radiant source with one peak wavelength at 222 nm as an example. The per-second ACGIH TLV at 222 nm is 0.7639 μJ/cm² as shown earlier. A controller of the present disclosure would operate this radiant source continuously such that the UV dosage received by an object is less than 0.7639 μJ/cm² per second. A benefit with a continuously operated radiant source is that it may be possible for a controller to be configured to operate the radiant source such that the per-second UV dosage received by an object approximates very closely to the per-second ACGIH TLV dosage without exceeding it.

Consider a scenario where a radiant source is mounting on the ceiling of a subway car or a bus. If a controller is configured to maximize the UV emission of the radiant source to the surface of a seat without exceeding ACGIH TLVs, then a passenger standing closer to the radiant source would receive a UV exposure much higher than the ACGIH TLVs, thus violating the ACGIH UV Safety Guidelines. On the contrary, if a controller is configured to maximize the UV emission of the radiant source to a standing passenger without exceeding ACGIH TLVs, then the UV dosage received by the surface of a seat may be too low to achieve an effective germicidal irradiation when the subway car is empty. The best solution in this scenario would be to use a distance sensor for measuring the distance of the closest object exposed to the radiant source (when there are a plurality of objects being irradiated by the radiant source), and then to have the controller maximizing the UV emission of the radiant source dynamically according to the distance (which is changing constantly) without violating ACGIH TLVs. In some embodiments, the apparatus further comprises a distance sensor. The distance sensor is configured to obtain periodically the distance of the closest object exposed to the UV radiation of the radiant source. The controller is configured to use the distance information provided by the distance sensor for adjusting or maximizing the radiant power emitted continuously by the radiant source such that the per-second UV dosage received by the closest object approximates, without exceeding, the per-second UV TLV dosage defined by the ACGIH Safety Guidelines. The closest object may change from time to time (e.g., due to movement of objects and/or the radiant source). For example, in the case of a subway car, the closest object may be a standing passenger, or a seating passenger, or a closest seat (when the subway car is empty). This condition can be represented in the following formula, where the distance D is the distance of the closest object provided by the distance sensor:

$${\sum }_{i = 190 nm}^{400 nm} \frac{Received Dosage at wavelength i at Distance D per second}{ACGIH TLV at wavelength i per second} ∼ 100 \%$$

When a UV radiant source only has one peak wavelength, then the formula above may be simplified to the following:

$$\frac{Received Dosage at the paeakwavelength at Distance D per second}{ACGIH TLV at the peak wavelength per second} ∼ 100 \%$$

Therefore, in some embodiments, the radiation source has a peak wavelength in a wavelength range 190˜400 nm, and the controller is configured to adjust or maximize the radiant power emitted continuously by the radiant source such that the per-second UV dosage received by the closest object at a distance D (provided by the distance sensor) approximates, without exceeding, a per-second UV TLV dosage calculated based on the ACGIH Safety Guidelines at the peak wavelength. Such embodiments when used in a subway car can maximize the germicidal irradiation according to the distance of the closest object to the radiant source without exceeding ACGIH TLV, even when the closest object and its distance both may change from time to time.

In some embodiments, the radiant source comprises one or more light emitting diodes (LEDs). The LED may have one peak wavelength, more than one peak wavelengths in a wavelength range 190˜400 nm, or no obvious peak wavelength at all. In some other embodiments, the radiant source includes an excimer lamp having a gas or combination of gases for producing a wavelength in a wavelength range 190˜400 nm, and the gas includes krypton-chloride (KrCl), krypton-bromine (KrBr), argon-fluorine (ArF), krypton-iodine (Krl), iodine (I₂), xenon-fluorine (XeF) gas, or their combination thereof. An excimer lamp with one gas type may only have one peak wavelength. When having two types of gas, an excimer lamp may have two peak wavelengths.

For the controller of the present disclosure to adjust the UV emission of the radiant light source in compliance with the ACGIH TLV, then it is necessary to have the ACGIH TLV data available. In some embodiments, the controller is configured to store the UV TLVs defined by the ACGIH, perhaps via a memory module. If a radiant source has only one peak UV wavelength, then the controller may be configured to only store the ACGIH TLV pertaining to this peak wavelength. Under this scenario, it is foreseeable to hardwire this peak-wavelength ACGIH TLV in the controller without using an explicit memory module.

When an object is at a distance from the radiant source, it is necessary for the controller to use the IES data for calculating the UV emission of the radiant source to maximize the UV dosage to the object (without exceeding the ACGIH TLV). Therefore, it is necessary for the controller to have access to the IES data of the radiant source. In some embodiments, the controller is configured to store an Illuminating Engineering Society (IES) data of the radiant source, perhaps via a memory module. It is foreseeable to hardcode the IES data in the controller circuitry without using an explicit memory module.

Sometimes it is preferrable to operate a radiant source to deliver a UV dosage much lower than the ACGIH TLV, e.g., when someone is overly sensitive to UV exposure. Therefore, in some embodiments, the present disclosure supports a mild mode operation wherein the controller is configured to operate the radiant source such that the UV dosage received by the object is at least 25% below the UV TLV dosage defined by the ACGIH. In other words, the controller enforces the following condition.

$${\sum }_{i = 190 nm}^{400 nm} \frac{Received Dosage Extropolated over 8 hours at wavelength i}{ACGIH TLV at wavelength i} < 75 \%$$

For a radiant source operating continuously, the above formula can be simplified to the following for the closest object at a distance D from the radiant source:

$${\sum }_{i = 190 nm}^{400 nm} \frac{Received Dosage at wavelength i at Distance D per second}{ACGIH TLV at wavelength i per second} < 75 \%$$

For a radiant source having only one peak wavelength and operating continuously, the above formula can be simplified to the following for the closest object at a distance D from the radiant source:

$$\frac{Received Dosage at the paeakwavelength at Distance D per second}{ACGIH TLV at the peak wavelength per second} < 75 \%$$

Sometimes it is preferrable to operate a radiant source to deliver a UV dosage much higher than the ACGIH TLV, e.g., for buses, subways, and elevators. In these environments, occupants come and go thus making these environments a potential infection hotspot. It would be reasonable to accelerate the germicidal irradiation for these environments by increasing the UL dosages above ACGIH TLVs, and not to worry about occupants being over-exposed with UV since they would never stay in these environments for eight hours. Therefore, in some embodiments, the present disclosure supports a boost mode operation wherein the controller is configured to operate the radiant source such that the UV dosage received by the object is at least 25% above the UV TLV dosage defined by the ACGIH. In other words, the controller enforces the following condition:

$${\sum }_{i = 190 nm}^{400 nm} \frac{Received Dosage Extropolated over 8 hours at wavelength i}{ACGIH TLV at wavelength i} > 125 \%$$

For a radiant source operating continuously, the above formula can be simplified to the following for the closest object at a distance D from the radiant source:

$${\sum }_{i = 190 nm}^{400 nm} \frac{Received Dosage at wavelength i at Distance D per second}{ACGIH TLV at wavelength i per second} > 125 \%$$

For a radiant source having only one peak wavelength and operating continuously, the above formula can be simplified to the following for the closest object at a distance D from the radiant source:

$$\frac{Received Dosage at the paeakwavelength at Distance D per second}{ACGIH TLV at the peak wavelength per second} > 125 \%$$

When an environment is not occupied, e.g., at night or during off hours, it would be reasonable to maximize the radiant power of a radiant source to have a deep sanitation of the environment over a short period of time such as 30 minutes or 60 minutes, or even longer (e.g., less than four hours). Therefore, in some embodiments, the present disclosure supports a full sanitation mode operation wherein the controller is configured to maximize a radiant power emitted by the radiant source over a short period of time.

It is foreseeable and in fact preferrable that some embodiments of the disclosed apparatus would support the regular mode, the mild mode, the boost mode, and/or the full sanitation mode and allow a user or a scheduler to switch from one mode to another.

In another aspect, the self-adjustable germicidal irradiation method includes (1) sensing the distance between a continuous radiant source capable of generating a wavelength in an ultraviolet (UV) wavelength range 190˜400 nm and an object, and (2) maximizing the UV dosage received by the object at the distance from the radiant source without exceeding UV TLV dosage defined by the ACGIH.

In some embodiments, the method includes (1) sensing the distance between a continuous radiant source capable of generating a wavelength in an ultraviolet (UV) wavelength range 190˜400 nm and the closest object exposed to the radiation of the radiant source, and (2) maximizing the UV dosage received by the closest object at the distance from the radiant source without exceeding UV TLV dosage defined by the ACGIH.

In some embodiments, the method of the present disclosure supports a mild mode operation wherein the radiant source is configured to operate at a radiant power such that the US dosage received by the object is at least 25% below the UV TLV dosage defined by the ACGIH.

In some embodiments, the method supports a boost mode operation wherein the radiant source is configured to operate at a radiant power such that the US dosage received by the object is at least 25% above the UV TLV dosage defined by the ACGIH.

In some embodiments, the method supports a full sanitation mode operation wherein the radiant source is configured to maximize its radiant power over a short period of time such as 30 minutes or 60 minutes, or even longer (e.g., less than four hours).

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 The Threshold Limit Values (dosage) according to ACGIH UV Safety Guidelines.

FIG. 2 schematically depicts a diagram of an embodiment using an excimer lamp and a motion sensor.

FIG. 3 shows an operation schedule of the first embodiment of the present disclosure.

FIG. 4 schematically depicts a diagram of another embodiment using an LED lamp and a distance sensor.

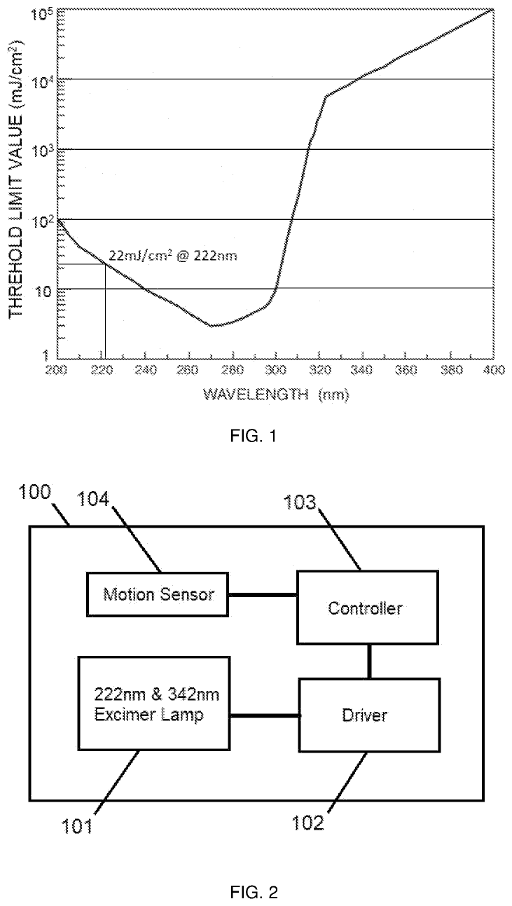

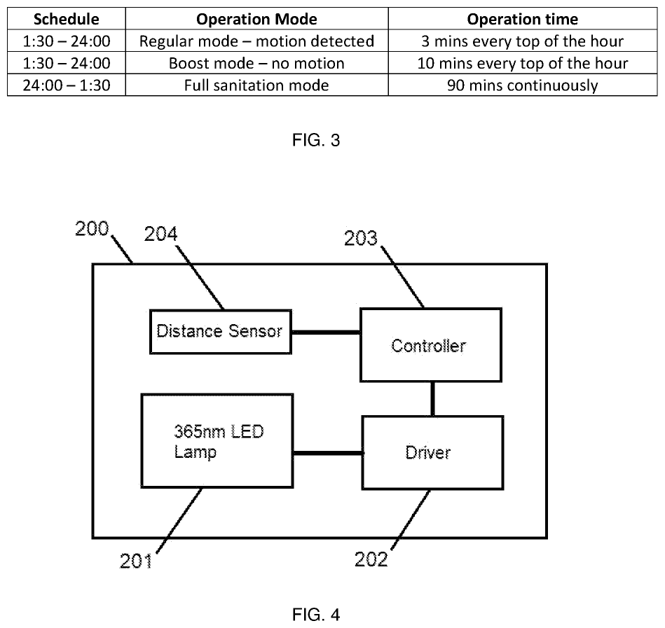

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of germicidal irradiation devices having different form factors.

The present disclosure apparatus includes a radiant source, a driver, and a controller. The driver converts an external power to an internal power to activate the radiant source. The connects to the driver and is configured to adjust a radiant power emitted by the radiant source. The radiation source is configured to generate a wavelength in an ultraviolet (UV) wavelength range 190˜400 nm. The controller is configured automatically to limit a UV dosage received by an object exposed to the UV radiation of the radiant source, such that the received UV dosage of the object does not exceed a UV threshold limit value (TLV) dosage defined by American Conference of Governmental Industrial Hygienists (ACGIH).

### Example Implementations

FIG. 2 is an embodiment of the self-adjustable germicidal irradiation apparatus of the present disclosure 100. The apparatus 100 includes an excimer lamp 101, a driver 102, a controller 103, and a motion sensor 104. The driver 102 converts an external power to an internal power for activating the excimer lamp 101. The excimer lamp includes two gases, krypton-chloride (KrCl) and iodine (I₂), thus having two peak wavelengths at 222 nm and 342 nm, respectively. Though not shown, the controller 103 has a fixed, built-in schedule for operating the excimer lamp as shown in FIG. 2. From 1:30-24:00, the controller would operate the excimer lamp intermittently, and the ON time would depend upon whether the motion sensor 104 detects any motion in the room or not. If there is a motion detected in the room, the controller will operate the excimer lamp in a regular mode where the excimer lamp is turned on for 3 minutes on top of every hour. If there is no motion detected in the room, the controller will operate the excimer lamp in a boost mode where the excimer lamp is turned on for 10 minutes on top of every hour. The UV dosage from the excimer lamp at both 222 nm and 342 nm wavelengths are hardcoded in the controller such that when the device is mounted at 10-ft ceiling, any occupant sitting in the room (10 ft-4.5 ft=5.5 ft away from the excimer lamp 101) will not receive more than ACGIH TLV dosage. When there is no motion detect, the controller operates the excimer lamp in a boost mode such that a desk that is 10 ft-2.5 ft=7.5 ft away from the excimer lamp will still receive a combined UV dosage from 222 nm and 342 nm wavelengths at 150% of the ACGIH TLV dosage. The IES data and the ACGIH TLV are hardcoded in the controller and reflected in the fixed mounting height (10-ft) of the apparatus and the operation on-time of the excimer lamp (3 minutes for a regular mode and 10 minutes for a boost mode). This embodiment also supports a full sanitation mode operation from 24:00 to 1:30, during which time the controller will operate the excimer lamp continuously for 90 minutes for thoroughly disinfecting the surroundings.

FIG. 4 is another embodiment of the present disclosure 200. The apparatus 200 includes an LED lamp 201, a driver 202, a controller 203, and a distance sensor 204. The driver 202 converts an external power to an internal power for activating the LED lamp 201. The LED lamp 201 has one peak wavelength at 365 nm. Though not shown, the IES data and the ACGIH TLVs are stored in the controller. Moreover, the controller 203 has a fixed, built-in schedule, also not shown. From 6:00 to 24:00, the controller will operate the LED lamp to deliver 200% of the per-second ACGIH TLV to the closest object (among a plurality of objects being irradiated) detected by the distance sensor 204, i.e., in a boost mode. While the distance of the closest object (e.g., a passenger in a subway car) to the LED lamp may change, or even the object itself may change (e.g., when the passenger leaves the subway car), the distance sensor will constantly update the distance data of the closest object to the controller. With the updated distance data, the controller uses the IES data and the ACGIH TLV data to calibrate the radiant power at which the LED lamp should operate in order to delivery 200% per-second ACGIH TLV dosage to the closest object. From 24:00 to 6:00, the controller will operate the LED lamp at its maximum radiant power, i.e., in a full sanitation mode. This embodiment may be used in a subway car or a commuter bus. The boost mode is set to delivery 200% ACGIH TLV to the closest object is acceptable for such environment for no passenger will stay in the say subway car or commuter bus longer than 4 hours. Therefore, the extrapolation of a passenger's received UV dosage over an 8-hour period will still fall below the ACGIH TLV (defined for 8-hour period).

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques. As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A self-adjustable germicidal irradiation apparatus, comprising
a radiant source;
a driver; and
a controller,
wherein:
the driver is configured to convert an external power to an internal power to activate the radiant source,
the controller is connected to the driver and is configured to adjust a radiant power emitted by the radiant source,
the radiation source is configured to generate a wavelength in an ultraviolet (UV) wavelength range of 190˜400 nm,
the controller is configured automatically to limit a UV dosage received by an object exposed to the UV radiation of the radiant source, such that the received UV dosage of the object when extrapolated over an eight-hour period does not exceed a UV threshold limit value (TLV) dosage defined by American Conference of Governmental Industrial Hygienists (ACGIH) for the wavelength.

2. The apparatus of claim 1, further comprising an occupancy sensor, wherein the controller is configured to toggle between two UV dosages received by the object exposed to the UV radiation of the radiant source, depending on whether there is a motion detected by the occupancy sensor.

3. The apparatus of claim 1, wherein the radiation source has one peak wavelength in the wavelength range of 190˜400 nm, and wherein the controller is configured to limit a peak-wavelength UV dosage received by the object exposed to the UV radiation of the radiant source not exceeding a UV TLV dosage calculated based on the ACGIH Safety Guidelines at the peak wavelength.

4. The apparatus of claim 1, wherein the radiation source has multiple wavelengths in the wavelength range of 190˜400 nm, and wherein the controller is configured to limit a sum of a ratio of the received UV dosage extrapolated over the eight-hour period by the object to an ACGIH TLV dosage for each wavelength in the 190˜400 nm range to be less than 100%.

5. The apparatus of claim 1, wherein the controller is configured to operate the radiant source continuously at a same radiant power, and wherein the controller is configured to limit a sum of a ratio of a per-second UV dosage received by the object to a per-second ACGIH TLV dosage for each wavelength in the 190˜400 nm range to be less than 100%.

6. The apparatus of claim 5, wherein the radiation source has one peak wavelength in the wavelength range of 190˜400 nm, wherein the controller is configured to operate the radiant source continuously, and wherein the controller is configured to limit the per-second UV dosage received by the object not to exceed the per-second ACGIH TLV dosage at the peak wavelength.

7. The apparatus of claim 5, further comprising a distance sensor, wherein the distance sensor is configured to obtain periodically a distance of a closest object exposed to the UV radiation of the radiant source, and wherein the controller is configured to use information of the distance provided by the distance sensor to adjust or maximize the radiant power emitted continuously by the radiant source such that the per-second UV dosage received by the closest object approximates, without exceeding, the per-second UV TLV dosage defined by the ACGIH Safety Guidelines.

8. The apparatus of claim 7, wherein the radiation source has a peak wavelength in a wavelength range 190˜400 nm, and the controller is configured to adjust or maximize the radiant power emitted continuously by the radiant source such that the per-second UV dosage received by the closest object approximates, without exceeding, a per-second UV TLV dosage calculated based on the ACGIH Safety Guidelines at the peak wavelength.

9. The apparatus of claim 1, wherein the radiant source comprises one or more light emitting diodes (LEDs).

10. The apparatus of claim 1, wherein the radiant source comprises an excimer lamp having a gas or a combination of a plurality of gases to produce a wavelength in the wavelength range of 190˜400 nm, and wherein the gas comprises krypton-chloride (KrCl), krypton-bromine (KrBr), argon-fluorine (ArF), krypton-iodine (Krl), iodine (I₂), xenon-fluorine (XeF) gas, or a combination thereof.

11. The apparatus of claim 1, wherein the controller is configured to store one or more UV TLVs defined by the ACGIH.

12. The apparatus of claim 1, wherein the controller is configured to store an Illuminating Engineering Society (IES) data of the radiant source.

13. The apparatus of claim 1, the apparatus supports a mild mode operation, wherein, when operating in the mild mode, the controller is configured to operate the radiant source such that the UV dosage received by the object is at least 25% below the UV TLV dosage defined by the ACGIH.

14. The apparatus of claim 1, the apparatus supports a boost mode operation, wherein, when operating in the boost mode, the controller is configured to operate the radiant source such that the UV dosage received by the object is at least 25% above the UV TLV dosage defined by the ACGIH.

15. The apparatus of claim 1, the apparatus supports a full sanitation mode operation, wherein, when operating in the full sanitation mode, the controller is configured to maximize the radiant power emitted by the radiant source over less than four hours.

16. A self-adjustable germicidal irradiation method, comprising:
sensing a distance between an object and a continuous radiant source capable of generating a wavelength in an ultraviolet (UV) wavelength range of 190˜400 nm; and
maximizing a UV dosage received by the object at the distance from the radiant source without exceeding a UV threshold limit value (TLV) dosage defined by American Conference of Governmental Industrial Hygienists (ACGIH).

17. The method of claim 16, wherein the object is a closest object among a plurality of objects that is closest to the radiant source.

18. The method of claim 16, wherein, when operating in a mild mode, the radiant source is configured to operate at a radiant power such that the UV dosage received by the object is at least 25% below the UV TLV dosage defined by the ACGIH.

19. The method of claim 16, wherein, when operating in a boost mode, the radiant source is configured to operate at a radiant power such that the UV dosage received by the object is at least 25% above the UV TLV dosage defined by the ACGIH.

20. The method of claim 16, wherein, when operating in a full sanitation mode, the radiant source is configured to maximize a radiant power over less than four hours.
